(19) United States
(12) Patent Application Publication
(10) Pub. No.: US 2006/0224093 A1
(43) Pub. Date: Oct. 5, 2006

(54) Method For Elevating The Comfort Level Of An Individual, Article Of Manufacture For Use In The Method, And Brace For Use With The Article In the Method

(76) Inventor: IMRE VIRAG, Toronto, Ontario (CA)

Correspondence Address: CONLEY ROSE, P.C., P. O. BOX 3267, HOUSTON, TX, 77253-3267, US

(21) Appl. No.: 11/278,681

(22) Filed: Apr. 5, 2006

(30) Foreign Application Priority Data
Apr. 5, 2005 (CA) 2503456

(51) Int. Cl. A61F 5/00

(52) U.S. Cl. 602/1

(57) ABSTRACT

A method for elevating the comfort level of an individual is disclosed and comprises the step of positioning: a crystal material proximate to the skin of said individual; and a film of metal over said skin, such that the crystal is disposed between the skin and film. Also disclosed is an article of manufacture comprising a metal film; a layer of transparent plastic sealed to said film; and a quantity of crystal, wherein the metal film and/or the plastic define one or more pockets and the crystal is disposed in each of said pockets. Also disclosed is a brace which comprises a layer of pliable material positioned adjacent the skin of an individual wearer in use and having said article secured thereto in use such that the crystal is proximate to the skin of the individual and disposed between said skin and film. The crystal material may be quartz.

### FIELD OF THE INVENTION

The present invention relates to the field of personal health and well being.

### BACKGROUND OF THE INVENTION

In the prior art, numerous therapeutic and medicinal uses of naturally-occurring crystals are found, U.S. Pat. No. 6,378,138, for example, teaches, inter alia, that quartz crystal enhances the crystalline properties of blood, and activates and enhances the pineal and pituitary glands. In view of these stated health benefits, this patent teaches the use of crystals for healing various bodily ailments. It is taught, for example, to utilize a stocking to which a crystal ornament can be detachably mounted. Crystal healing elements, as well, can be placed in a pouch, and worn on the body of a user.

### SUMMARY OF THE INVENTION

According to one aspect, the present invention comprises a method for elevating the comfort level of an individual. This method comprises the step of positioning a crystalline material proximate to the skin of said individual and positioning a film of metal over said skin such that the crystalline material is disposed between said skin and said film

According to another aspect, the present invention comprises an article of manufacture. The article comprises: a film of metal; a layer of transparent plastic sealed to said metal film and defining one or more pockets; and a quantity of crystalline material disposed in each of said one or more pockets.

According to another aspect, the present invention comprises a wearable brace for use with the article of manufacture. The brace comprises a layer of pliable material having an inner surface, said surface being disposed against the skin of an individual in use and having said article of manufacture secured thereto such that the crystalline material is proximate to the skin of the individual and disposed between said skin and said film.

Other advantages, features and characteristics of the present invention, as well as methods of operation and functions of the related elements of the structure, and the combination of parts and economies of manufacture, will become more apparent upon consideration of the following detailed description and the appended claims with reference to the accompanying drawings, the latter being briefly described hereinbelow

### BRIEF DESCRIPTION OF THE DRAWINGS

FIG. 1 is a perspective view of a preferred embodiment of the article of manufacture of the invention;

FIG. 2 is a top plan view of the article of FIG. 1;

FIG. 3 is a view along section 3-3 of FIG. 2;

FIG. 4 is a perspective view of a preferred embodiment of the brace of the invention;

FIG. 5 is a view of the structure of FIG. 4 in a partially-fastened position;

FIG. 6 is a view of the structure of FIG. 4 in a fully-fastened position;

FIG. 7 is a view along section 7-7 of FIG. 4; and

FIG. 8 is a view of the brace of FIG. 4 in use

### DETAILED DESCRIPTION

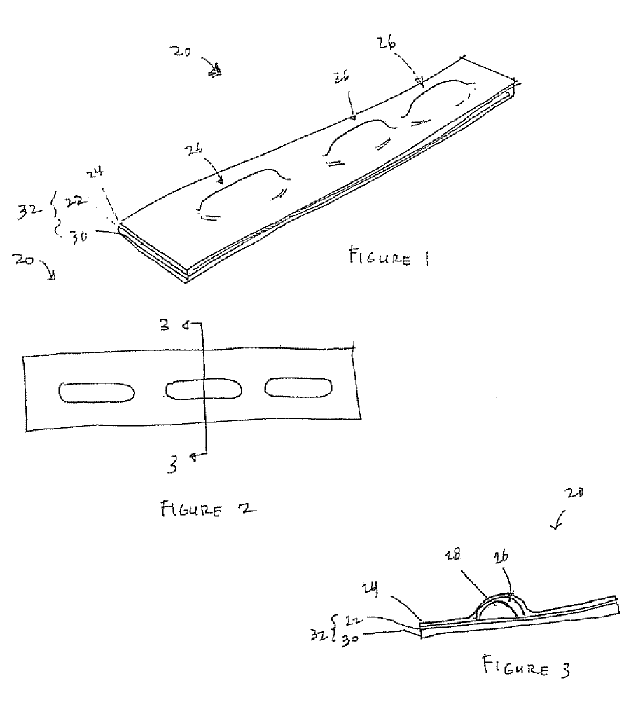

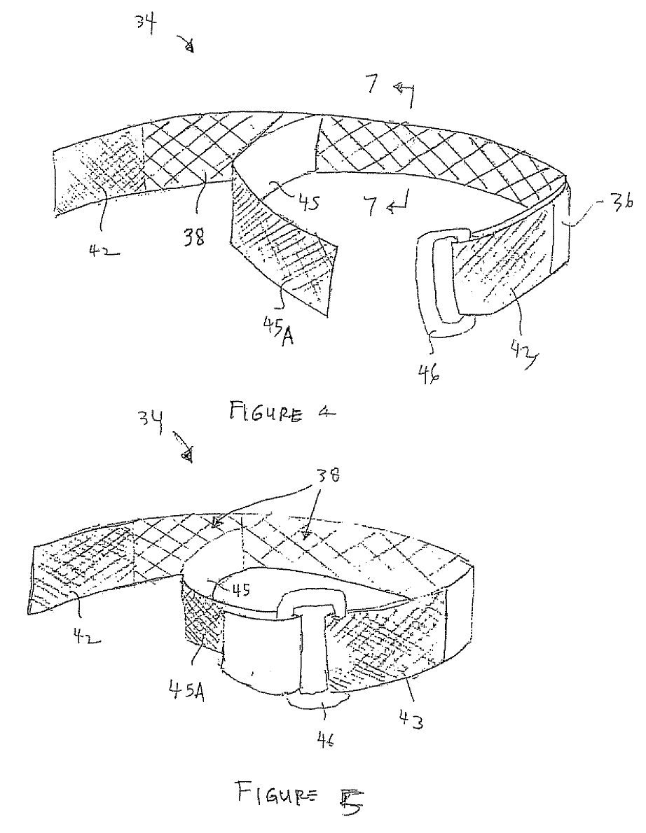

With general reference to FIGS. 1, 2 and 3, the preferred embodiment of the article of manufacture which forms part of the present invention is illustrated and designated by general reference numeral 20.

The article 20 comprises a film of metal 22 and a layer of flexible transparent plastic 24 sealed to said metal film 22. The metal 22 and/or layer of plastic 24 define one or more, more particularly, a plurality of, pockets 26, spaced-apart from one another. The article 20 further comprises a quantity of crystalline material 28 disposed in each of said one or more pockets 26,

The film of metal 22 forms part of a flexible layer 32 of metallized oriented polypropylene (MOP) which includes, in addition to said metal film, a substrate layer 30 of oriented polypropylene.

The transparent plastic 24 is transparent asymmetrical bi-oriented polypropylene, heat sealed to the MOP 32.

The crystalline material 28 is a particulate quartz material, having grain size between 001 and 0.003 inches.

An exemplary preferred embodiment of the brace of the present invention is shown in FIGS. 4-8, in combination with an operatively disposed article 20 and designated with general reference numeral 34.

As best seen in FIG. 7, the brace 34 includes a layer 36 of nylon material and a mesh panel 38 secured to said pliable layer 36 to form a pocket 40 in which said article 20 is disposed.

The layer of nylon material 36 in this case takes the form of a strip having a loop 46 disposed on one end thereof. A panel 43 of hook and loop fastening material is disposed at the one end of the strip, on the side thereof opposite the mesh layer 38. A panel 42 of hook and loop fastening material is disposed at the other end of the strip, on the side common with the mesh layer 38. A strip 45 extends from layer 36 intermediate the ends, one side thereof 45A being faced with hook and loop material and orientated such that, when strip 45 overlies the other end of the strip, panel 42 presents towards the hook and loop material of strip 45.

A partially-fastened configuration of brace 34 is shown in FIG. 5A In this configuration, strip 45 is threaded through loop 46 and fastened upon itself.

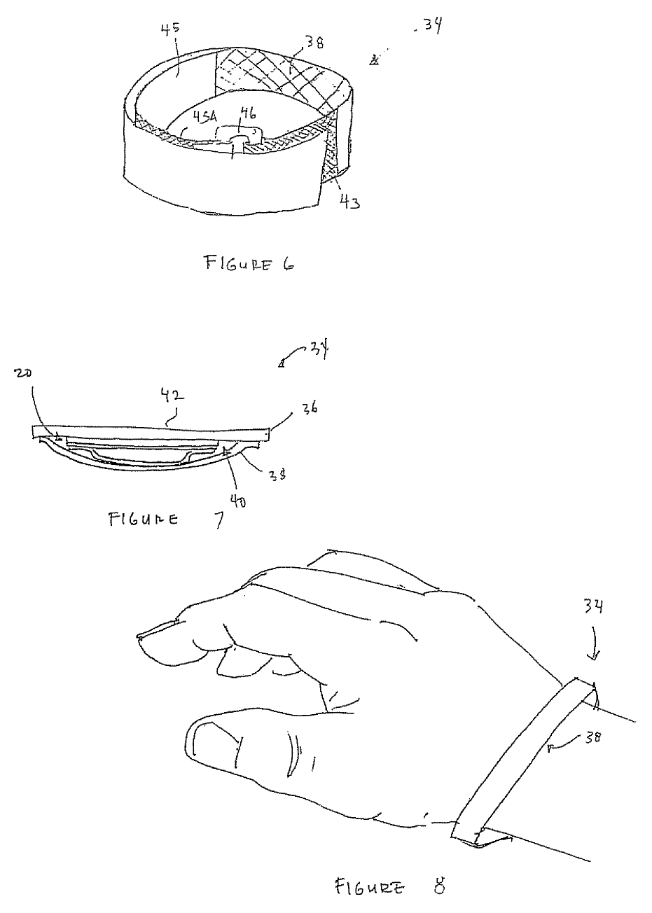

A fully-fastened configuration of brace 34 is shown in FIG. 6. In this configuration, the other end of layer 36 is drawn over the loop 36, with panel 42 engaging panel 43 and the hook and loop material of strip 45.

In use as shown in FIG. 8, the brace 34 is positioned such that the mesh layer 38 is adjacent the skin of the individual wearer; the layer of transparent plastic 24 lies atop the mesh 38; the crystalline material 28 lies atop the transparent plastic 24; the metal film 22 overlies the crystalline material 28, and the nylon overlies the metal film 22.

Use in this manner elevates the general comfort level of the individual wearer.

Without intending to be bound by theory, it is believed that the vibratory resonance of the crystalline material, disposed in close proximity to the body, offers a steady rhythm to the body of the wearer, which rhythm provides a measure or tempo to which the natural functions of the body self-regulate, thereby to improve performance on a micro level, in the same manner that a steady beat or rhythm can improve macro level human performance, as is well established in the fields, for example, of music and athletics

Again, without intending to be bound by theory, it is believed that the metallic film 22 serves as a reflector, and the substrate layer 30 serves as an insulator, for said vibrations, to direct same towards the body in use.

The rhythm may advantageously be focused to areas of the body which are not functioning normally, such as inflamed tissues or painful joints, Thus, the brace can advantageously be made suitable for enveloping at least a portion of at least one of: a finger, a hand, a wrist (as shown), an elbow, a shoulder, an arm, a head, a scalp, a face, a neck, a torso, a back, at least one breast, a waist, a groin, a hip, a leg, a knee, an ankle, a foot and a toe, and specifically may take the form of a wound dressing garment, a finger wrap, a headband, a wristband, a sleeve, a vest, a jacket, a coat, a shirt, a sweater, a neck tube, a girdle, shorts, pants, leggings, a leg wrap, a toe wrap, and an undergarment.

The details of construction of braces embodying the above functionality are readily understood by persons of ordinary skill in the art and as such are not detailed herein.

Whereas but a single preferred embodiment of the article, brace and method are described herein, it will be evident that various modifications may be made

For example, whereas the crystalline material is indicated to be a particulate material having a grain size of 0.001-0.003 inches, non-particulate crystalline material can be used, as can particulate material of other grain size. As well, whereas the crystalline material is indicated to be quartz, other crystalline materials can be used, including, but not limited to Agate; Alexandrite; Amazonite; Amber; Amethyst; Apatite; Aquamarine; Aventurine; Azurite; Azurite/Malachite; Black Obsidian; Calcite; Carnelian; Celestite; Chrysophase; Chrysocolla; Citrine; Diamond; Emerald; Fire Agate; Flourite; Galena; Garnet; Hematite; Jade; Jasper; Kunzite; Lapis Lazuli; Lepidolite; Malachite; Moonstone; Onyz; Opal; Pearl; Peridot; Pytie; Rhodochrosite; Rose Quartz; Ruby; Rutilated Quartz; Sapphire; Selenite; Smokey Quartz; Sodalite; Sugalite; Topaz; Tiger Eye; Tourmalated Quartz; Tourmaline; Turquoise and Variscite.

Further, whereas asymmetric bi-oriented polypropylene is utilized to form a transparent layer, other transparent plastics could be utilized and, indeed, transparent plastic could readily be avoided altogether if, for example, crystals were adhered directly to the metal layer (in which case it would be important to ensure that abrasion was not a concern, for example, by smoothing the crystal surface)

As well, whereas a mesh layer is taught in the brace, mesh could be avoided if, for example, the as sewed or otherwise adhered to the pliable layer

Additionally, metal films other than metallized oriented polypropylene, and pliable layers other than nylon, could readily be substituted. Neoprene, for example, could readily be substituted for nylon, as readily understood by persons of ordinary skill in the art.

Accordingly, it will be understood that the scope of the present invention is to be limited only by the claims appended hereto, purposively construed.

1. A method for elevating the comfort level of an individual, said method comprising the step of positioning:
a crystalline material proximate to the skin of said individual; and
a film of metal over said skin, such that the crystalline material is disposed between said skin and said film.

2. A method according to claim 1, wherein the crystalline material is a particulate crystalline material.

3. A method according to claim 1, wherein the crystalline material is selected from the group consisting of Agate; Alexandrite; Amazonite; Amber; Amethyst; Apatite; Aquamarine; Aventurine; Azurite; Azurite/Malachite; Black Obsidian; Calcite; Carnelian; Celestite; Chrysophase; Chrysocolla; Citrine; Diamond; Emerald; Fire Agate; Flourite; Galena; Garnet; Hematite; Jade; Jasper; Kunzite; Lapis Lazuli; Lepidolite; Malachite; Moonstone; Onyz; Opal; Pearl; Peridot; Pytie; Quartz; Rhodochrosite; Rose Quartz; Ruby; Rutilated Quartz; Sapphire; Selenite; Smokey Quartz; Sodalite; Sugalite; Topaz; Tiger Eye; Tourmalated Quartz; Tourmaline; Turquoise and Variscite.

4. A method according to claim 1, wherein the film of metal forms part of a layer of metallized oriented polypropylene.

5. A method according to claim 1, wherein, in the positioning step, a layer of transparent plastic that is sealed to the metal film to sandwich the crystalline material is placed proximate said skin.

6. A method according to claim 5, wherein the transparent plastic is transparent asymmetrical bi-oriented polypropylene.

7. A method according to claim 1, wherein, in the positioning step, the crystalline material is placed proximate to inflamed tissues of the individual

8. A method according to claim 1, wherein, in the positioning step, the crystalline material is placed proximate to a painful joint of the individual.

9. An article of manufacture comprising:
a film of metal; a layer of transparent plastic sealed to said metal film; and
a quantity of crystalline material, wherein the film of metal and/or the layer of plastic define on or more pockets and wherein the crystalline material is disposed in each of said one or more pockets.

10. An article according to claim 9, wherein the crystalline material is a particulate crystalline material.

11. An article according to claim 91 wherein the crystalline material is selected from the group consisting of: Agate; Alexandrite; Amazonite; Amber; Amethyst; Apatite; Aquamarine; Aventurine; Azurite; Azurite/Malachite; Black Obsidian; Calcite; Carnelian; Celestite; Chrysophase; hrysocolla; Citrine; Diamond; Emerald; Fire Agate; Flourite; Galena; Garnet; Hematite; Jade; Jasper; Kunzite; Lapis Lazuli; Lepidolite; Malachite; Moonstone; Onyz; Opal; Pearl; Peridot; Pytie; Quartz; Rhodochrosite; Rose Quartz; Ruby; Rutilated Quartz; Sapphire; Selenite; Smokey Quartz; Sodalite; Sugalite; Topaz; Tiger Eye; Tourmalated Quartz; Tourmaline; Turquoise and Variscite.

12. An article according to claim 9, wherein the film of metal forms part of a layer of metallized oriented polypropylene.

13. An article according to claim 9, wherein the transparent plastic is transparent asymmetrical bi-oriented polypropylene

14. An article according to claim 9, wherein said one or more pockets comprises a plurality of pockets, spaced-apart from one another.

15. A wearable brace for use with the article of claim 9, comprising:
a layer of pliable material positioned adjacent the skin of an individual wearer in use and having said article secured thereto in use such that the crystalline material is proximate to the skin of the individual and disposed between said skin and said film.

16. A brace according to claim 15, further comprising;
a mesh panel secured to said layer of pliable material to form a pocket in which said article is disposed in use.

17. A brace according to claim 15, in the form of a wound dressing garment, a finger wrap, a headband, a wristband, a sleeve, a vest, a jacket, a coat, a shirt, a sweater, a neck tube, a girdle, shorts, pants, leggings, a leg wrap, a toe wrap, and an undergarment.

18. A brace according to claim 15, wherein the brace is suitable for enveloping at least a portion of at least one of: a finger, a hand, a wrist, an elbow, a shoulder, an arm, a head, a scalp, a face, a neck, a torso, a back, at least one breast, a waist, a groin, a hip, a leg, a knee, an ankle, a foot and a toe.
